(19) United States
(12) Patent Application Publication
(10) Pub. No.: US 2025/0111905 A1
(43) Pub. Date: Apr. 3, 2025

(54) METHOD AND SYSTEM FOR PREDICTING DRUG-DRUG INTERACTIONS

(71) Applicant: B. G. NEGEV TECHNOLOGIES AND APPLICATIONS LTD., Beer Sheva (IL)

(72) Inventors: Guy SHTAR, Beer Sheva (IL); Adir SOLOMON, Beer Sheva (IL); Eyal MAZUZ, Beer Sheva (IL); Lior ROKACH, Beer Sheva (IL); Bracha SHAPIRA, Beer Sheva (IL)

(73) Assignee: B. G. NEGEV TECHNOLOGIES AND APPLICATIONS LTD., Beer Sheva (IL)

(21) Appl. No.: 18/834,668

(22) PCT Filed: Feb. 2, 2023
(86) PCT No.: PCT/IL2023/050116

§ 371 Date: Jul. 31, 2024

Related U.S. Application Data
(60) Provisional application No. 63/305,785, filed on Feb. 2, 2022.
(60) Provisional application No. 63/355,859, filed on Jun. 27, 2022.

(51) Int. Cl. G16C 20/30; G16C 20/70

(52) U.S. Cl. CPC G16C 20/30; G16C 20/70

(57) ABSTRACT

A method and system of predicting drug-drug interactions (DDIs) may include receiving: (a) a DDI data structure, representing known DDIs between baseline drugs, (b) a plurality of baseline drug data elements, comprising line-notation descriptions of chemical structure of corresponding baseline drugs, and (c) a substance data element, comprising a line-notation description of a chemical structure of a substance of interest. Embodiments may calculate one or more similarity metric values, representing structural similarity between the substance of interest and specific baseline drugs, and select a subset of the baseline drugs based on the one or more similarity metric values. Embodiments may subsequently predict DDI between the substance of interest and a target baseline drug, based on (i) the selected subset of baseline drugs and (ii) the DDI data structure.

### CROSS-REFERENCE TO RELATED APPLICATIONS

This application claims the benefit of priority of U.S. Provisional Application Nos. 63/305,785, filed Feb. 2, 2022, titled “Machine learning models for predicting preclinical drug-drug interactions using chemical structure” and 63/355,859, filed Jun. 27, 2022, titled “METHOD AND SYSTEM FOR PREDICTING DRUG-DRUG INTERACTIONS”. The contents of these applications are all incorporated herein by reference in their entirety.

### FIELD OF THE INVENTION

The present invention relates generally to machine-learning (ML) based prediction of chemical or biological interactions. More specifically, the present invention relates to methods and systems for predicting preclinical Drug-Drug Interactions (DDIs).

### BACKGROUND OF THE INVENTION

Drug-drug interactions (DDIs) are a critical component of drug safety surveillance, allowing effective and safe treatment of comorbidity. Laboratory studies aimed at detecting DDIs are typically difficult, expensive, and time-consuming, and therefore the development of in-silico methods is critical.

Machine Learning (ML) based approaches for DDI prediction have been developed. However, in many cases, the ability of such solutions to achieve significant accuracy relies on extraction of data that is only available towards the end of the drug's expensive development process, e.g., following conduction of clinical trials.

Other methods of DDI prediction rely on intricate, iterative analysis of the molecular structures of drugs of interest. It has been observed by the inventors that such analysis typically yields a cumbersome, and often prohibitively large representation of the structure of the underlying drugs. Subsequent application of ML models on such large representations of molecular structures typically leads to overfitting of the ML models, and results in poor performance when inferring the ML models on new data samples.

Additional ML based techniques for predicting DDI between drugs of interest may utilize previously obtained information regarding DDI between a drug of interest and a first baseline drug, to gain insight regarding DDI between the drug of interest and a second baseline drug. However, such techniques may not be applied on new drugs, for which no DDI information is available.

### SUMMARY OF THE INVENTION

Embodiments of the invention may include a method of predicting drug-drug interactions (DDIs), by at least one processor.

According to some embodiments, the at least one processor may be configured to receive a DDI data structure, wherein each entry (i,j) represents a known DDI between a first baseline drug (i) and a second baseline drug (j) of a plurality of baseline drugs; receive a plurality of baseline drug data elements, each including a line-notation description of a chemical structure of a corresponding baseline drug of the plurality of baseline drugs; receive a first substance data element, including a line-notation description of a chemical structure of a respective first substance of interest; calculate one or more first similarity metric values, representing a structural similarity between the first substance of interest and a specific baseline drug, based on the first substance data element and the relevant baseline drug data element; select a first subset of the plurality of baseline drugs, based on the one or more first similarity metric values; predict a DDI between the first substance of interest and a target baseline drug of the plurality of baseline drugs, based on (a) the first selected subset of baseline drugs and (b) the DDI data structure.

According to some embodiments, the at least one processor may be further configured to receive a second substance data element including a line-notation description of a chemical structure of a second substance of interest; calculate one or more second similarity metric values, each representing a structural similarity between the second substance of interest and a specific baseline drug, based on the second substance data element and the relevant baseline drug data element; select a second subset of the plurality of baseline drugs, based on the one or more second similarity metric values; and predict a DDI between the first substance of interest and the second substance of interest, based on (a) the first subset of baseline drugs, (b) the second subset of baseline drugs, and (c) the DDI data structure.

According to some embodiments the line-notation description article provided by inventors a Structure Simplified Molecular Input Line Entry System (SMILES) representation.

According to some embodiments, the similarity metric values may be, for example a Tanimoto similarity value, an edit distance similarity value, a Longest Common Subsequence (LCS) similarity value, a Normalized Longest Common Subsequence (NLCS) similarity value, and a Term frequency (TF) similarity value. Additionally, or alternatively, the at least one processor may be further configured to calculate two or more first similarity metric values, selected from the abovementioned a group, based on a SMILES representations of the specific baseline drug data element and the first substance data element; and calculate a similarity metric value, as a function (e.g., a weighted average) of the two or more first similarity metric values.

According to some embodiments, the at least one processor may be configured to calculate a similarity metric value between a new drug and a baseline drug by calculating an edit value, representing a number of edit operations that are required in order to convert the new drug data element to the baseline drug data element; and normalizing the edit value by at least one of a length of the new drug data element and the baseline drug data element.

According to some embodiments, the at least one processor may be configured to predict occurrence of DDI between the first substance of interest and the target baseline drug by applying a machine-learning algorithm to calculate one or more DDI scores, representing an expected DDI between one or more baseline drugs of the first subset of baseline drugs, and the target baseline drug, based on the DDI data structure; and predicting DDI between the first substance of interest and the target baseline drug based on the one or more DDI scores.

Additionally, or alternatively, the at least one processor may be configured to predict occurrence of DDI between the first substance of interest and the second substance of interest by applying a machine-learning algorithm to calculate one or more DDI scores, representing an expected DDI between one or more baseline drugs of the first subset of baseline drugs, and one or more respective baseline drugs of the second subset of baseline drugs, based on the DDI data structure; and predicting DDI between the first substance of interest and the second substance of interest based on the one or more DDI scores.

According to some embodiments, the at least one processor may be configured to calculate a DDI score representing an expected DDI between a first baseline drug and a second baseline drug by extracting, from the DDI data structure, a first DDI embedding vector, representing known DDIs between the first baseline drug and other baseline drugs of the plurality of baseline drugs in an embedding space; extracting, from the DDI data structure, a second DDI embedding vector, representing known DDIs between the second baseline drug and other baseline drugs of the plurality of baseline drugs in the embedding space; applying a vector operation on the first DDI embedding vector and the second DDI embedding vector, to produce a DDI embedding vector; and applying a machine-learning model on the product DDI embedding vector, to obtain the DDI score.

Additionally, or alternatively, the at least one processor may be configured to extract a DDI embedding vector of a specific baseline drug from the DDI data structure by: extracting an interim vector of the DDI data structure that corresponds to the specific baseline drug; and applying an embedding algorithm on the extracted interim vector to obtain the DDI embedding vector of the specific baseline drug.

Additionally, or alternatively, the at least one processor may be configured to determine a drug regimen, that includes at least the first substance of interest, based on the predicted occurrence of DDI; and produce a regimen recommendation data element, representing the determined regimen.

Embodiments of the invention may include a system for predicting occurrence of DDIs. Embodiments of the system may include a non-transitory memory device, wherein modules of instruction code are stored, and at least one processor associated with the memory device, and configured to execute the modules of instruction code.

Upon execution of said modules of instruction code, the at least one processor may be configured to: receive a DDI data structure, wherein each entry (i,j) represents a known DDI between a first baseline drug (i) and a second baseline drug (j) of a plurality of baseline drugs; receive a plurality of baseline drug data elements, each may include a line-notation description of a chemical structure of a corresponding baseline drug of the plurality of baseline drugs; receive a first substance data element, may include a line-notation description of a chemical structure of a first substance of interest; calculate one or more first similarity metric values, each representing a structural similarity between the first substance of interest and a specific baseline drug, based on the first substance data element and the relevant baseline drug data element; select a first subset of the plurality of baseline drugs, based on the one or more first similarity metric values; and apply a machine-learning algorithm to predict a DDI between the first substance of interest and a target baseline drug of the plurality of baseline drugs, based on (a) the first selected subset of baseline drugs and (b) the DDI data structure.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic pseudo-code description of an LAMFP algorithm, according to some embodiments of the invention;

FIG. 2 is a block diagram, depicting a computing device which may be included in a system for predicting Drug-Drug Interactions (DDIs) according to some embodiments of the invention;

FIGS. 3A and 3B are block diagrams, depicting two exemplary applications of a system for predicting DDI, according to some embodiments of the invention;

FIG. 4 is a block diagram, depicting modules of a system for predicting DDI, according to some embodiments of the invention; and

FIG. 5 is a flow diagram, depicting a method of predicting DDI, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

As elaborated herein, embodiments of the invention may provide a simplified, similarity-based method and system for preclinical DDI prediction of new drugs of interest.

The term “simplified” may be used in this context to indicate that embodiments of the invention may employ small-scale representations of chemical structures, to easily identify characteristics of similarity between different drug molecules, and apply ML models on these representations while avoiding the effect of overfitting, as was experimentally observed with large-scale structural representations.

The term “preclinical” may be used in this context to indicate that embodiments of the invention may perform DDI prediction without requiring data that can only be obtained during, or following clinical trials (e.g., where only chemical structure data of the drug of interest is available).

As elaborated herein, the DDI prediction system of the present invention may be configured to evaluate a structural similarity between new, unseen drugs of interest and baseline drugs, for which DDI information is already available, and infer the available DDI information on the new drugs of interest to predict DDI with one or more baseline drugs.

Additionally, or alternatively, the DDI prediction system of the present invention may infer the available DDI information on a pair of new drugs of interest, to predict occurrence of DDI between the two drugs of interest in that pair, as elaborated herein.

In recent years, researchers have gathered drug data from literature, reports, and other sources to create databases that can aid in developing in-silico DDI prediction methods.

As a result, machine learning approaches for DDI prediction have gained popularity, saving time and money. These methods can be categorized into two groups: (1) preclinical DDI prediction: methods which use the chemical structure of a drug as input; and (2) modality-intensive DDI prediction: methods that use a single domain-expert-engineered drug feature (or fuse several of these features), such as the known drug-drug interactions, drug-target interactions, and side effects of a given drug, to predict its DDIs.

The main limitation of modality-intensive DDI prediction methods stems from the fact that the required domain-expert engineered drug features are not available until the advanced stages of the drug lifecycle. Thus, the predictions of models based on these features are only available after the drug has been clinically tested or even approved. Furthermore, modality intensive DDI prediction requires significant human resources, time, and effort.

Therefore, embodiments of the invention may focus on the preclinical DDI prediction task, a task which is quite challenging due to the lack of handcrafted features.

Experimental results have shown that known DDIs are very accurate predictors of new interactions, and outperform currently available modality-intensive methods which incorporate many drug features. By using known DDIs to predict unknown ones, the problem can be tackled as a classical link prediction problem and solved using matrix factorization (MF) techniques.

This solution is analogous to collaborative filtering in recommender systems, where calculating a recommendation for a user is done by collecting information about users with similar taste and preferences; collaborative filtering is usually performed using MF techniques, which in general perform better than methods that use content-based information or meta-data regarding the items and users.

Embodiments of the invention may employ MF as part of DDI prediction. From a recommender system's perspective, the preclinical DDI prediction task is similar to the cold-start task, facing the same challenges created by insufficient data.

Embodiments of the invention may solve the cold-start recommendation system problem by employing an Adjacency Matrix Factorization with Propagation (AMFP) algorithm, adapted to uses known DDIs to predict new ones.

Embodiments of the invention may introduce a Lookup Adjacency Matrix Factorization with Propagation (LAMFP) algorithm, which may perform matrix factorization on the adjacency matrix (also referred to herein as DDI data structure) and propagate each drug's representation to interacting drugs.

LAMFP may deal with unseen drugs by employing a simple similarity-based mechanism, referred to as a “lookup mechanism”, to replace unseen drugs with known drugs.

Currently available methods of preclinical DDI prediction were reported to perform well on a holdout evaluation scheme, however they struggle when faced with unseen drugs.

Embodiments of the invention may leverage the molecular structure, which is available at any stage of the drug development process, to predict occurrence of DDI by processing the interactions of chemically similar drugs.

Experimental results have shown that embodiments of the invention outperforms state-of-the-art solutions and complex deep learning architectures like directed message passing neural networks for the preclinical drug-drug interaction prediction task. It has been experimentally observed that methods which model the molecular structure as a graph of atoms and bonds, underperform compared to the straightforward, similarity-based method of the present invention when evaluating DDIs involving new, unseen substances of interest.

Embodiments of the invention may implement the LAMFP algorithm, to support unseen drugs, by performing a lookup on existing drugs, based on their chemical structure.

Additionally, or alternatively, embodiments of the invention may assess the performance of various chemical structure similarity metrics for the task of DDI prediction and may provide an ensemble of chemical structure similarity metrics that may be optimal for DDI prediction.

Embodiments of the invention may evaluate several preclinical DDI prediction methods based on recurrent neural networks (RNNs) and message-passing neural networks.

Embodiments of the invention may formulate the preclinical drug-drug interaction prediction problem as a binary classification problem: Given an existing drug i, embodiments may use its chemical structure s(i). Similarly, embodiments may use the chemical structure for new drugs j and l, represented by s(j) and s(l) respectively. Embodiments may predict whether an interaction will exist between (1) drugs i and j based on their chemical structure, denoted by interaction(s(i); s(j)), and (2) new drugs j and l, based on their chemical structure denoted by interaction(s(j); s(l)).

According to some embodiments, the interaction prediction could be one of the following two types of interactions: (1) an interaction that exists, where interaction(s(⋅); s(⋅))=1, or (2) an interaction does not exist, where interaction(s(⋅); s(⋅))=0. The adjacency matrix factorization with propagation (AMFP) algorithm was developed for DDI prediction, based on factorization of the interaction graph adjacency matrix (also referred to as DDI data structure).

As known in the art, matrix factorization techniques are widely used in recommender systems, where each user and item are represented by a compressed latent vector (embedding).

In AMFP, each drug is represented by an embedding, which is used to reconstruct the interaction network. To calculate the drug's embedding, embodiments may first represent all drug interactions with an adjacency matrix (DDI data structure) such that it holds all of the known interactions between all drugs. Embodiments may subsequently use an inner product calculation between all drugs vectors (e.g., for each row i and each column j):

$${\hat{y}}_{i , j} = {\sum }_{w = 1}^{k} {p}_{iw} {q}_{jw}$$

where the embeddings of drugs i and j are positioned in a shared vector space (shared weights), expressed by pᵢ and qⱼ, and the embeddings' size is determined by the parameter k.

Since low k values may cause underfitting, and large k values can lead to overfitting, embodiments may improve the inner product calculation using the following calculation:

$${\hat{y}}_{i , j} = μ + {b}_{i} + {b}_{j} + {\sum }_{w = 1}^{k} {p}_{iw} {q}_{jw}$$

such that bᵢ and bⱼ stand for the bias values for drugs i and j respectively.

The μ value may be calculated based on the average value of the entire adjacency matrix.

These parameters may be optimized using the stochastic gradient descent technique with a binary cross-entropy loss.

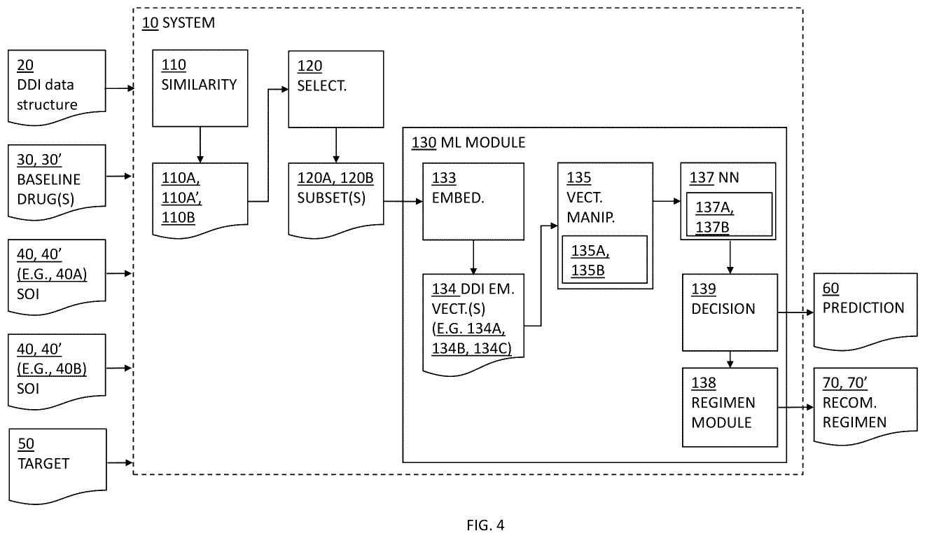

Embodiments of the invention may implement the AMFP algorithm with a neural network (e.g., NN 137 of FIG. 4), as elaborated herein. AMFP was designed to learn a drug representation from existing interactions, however this assumption may render AMFP unsuitable for new drugs where no existing interactions are known. In other words, the AMFP algorithm may be vulnerable to the cold-start problem.

Due to AMFP's strong performance, its simplicity in using a simple, single input of existing DDIs and its ability to support small molecules and biologics with a single model, embodiments may extend it to tackle the drawback of inability to support unseen drugs, resulting in the LAMFP algorithm.

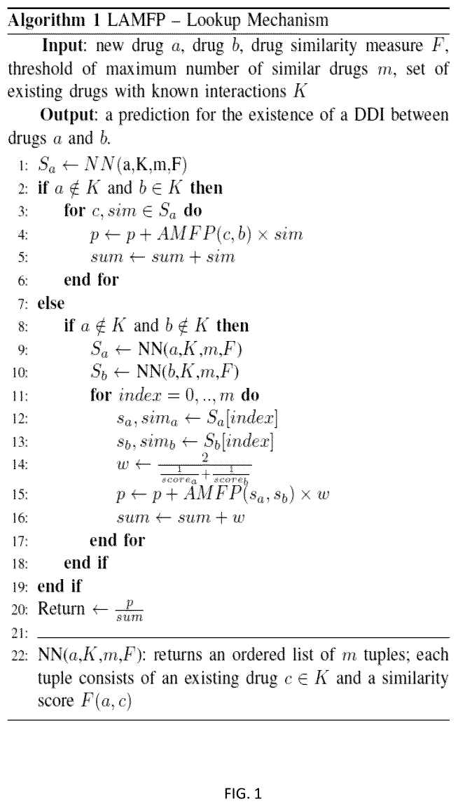

Reference is now made to FIG. 1, which presents a schematic pseudo-code description of the LAMFP algorithm, according to some embodiments of the invention.

As shown in FIG. 1, the LAMFP algorithm may uses a lookup mechanism in which the prediction of unseen drugs is calculated using the predictions of m known drugs. The predictions may be based on a similarity measure denoted by F.

Embodiments of the invention may define two cases for LAMFP: (1) predicting drug interaction involving a single new, unseen molecule: interaction(s(i); s(j))), and (2) predicting drug interaction involving two new molecules which: interaction(s(j); s(l)).

In the case of the former, embodiments may calculate the new drug's prediction by finding the weighted average of the predictions of the m most similar known drugs' based on F.

In the case of the latter, the prediction is calculated for two unseen drugs. Therefore, based on F, embodiments may retrieve the most similar m known drugs for s(j) and s(l)). Embodiments may subsequently calculate the weighted mean prediction between each two corresponding drugs in the two lists. Weights may be found by calculating the harmonic mean of the similarity scores.

In cases in which both molecules are known, the LAMFP algorithm uses the AMFP algorithm (i.e., without the lookup mechanism) to provide DDI predictions. The lookup mechanism may rely on the test-time augmentation (TTA) technique, which was shown to be beneficial for creating robust and accurate machine learning models.

The use of this mechanism may enable AMFP to handle unseen drugs and provide more accurate DDI predictions.

According to some embodiments, LAMFP may support various similarity metrics. In order to examine various similarity metric values (denoted as F in FIG. 1, and 110A/110B of FIG. 4).

For example, a similarity metric value 110A may include Tanimoto Similarity: a similarity metric values which is given by calculating the 2048-bit Morgan fingerprints and determining the proportion of shared chemical substructures.

In another example, a similarity metric value 110A may include an edit distance (ED) value. Embodiments may calculate a minimal number of edit operations (e.g., insertion, deletion, and substitution) to convert s(i) to s(j) and denote it as edit (s(i); s(\hat{ }j)). Embodiments may subsequently calculate the similarity metric value 110A according to the following equation:

$$EditSim ( s ( i ) , s ( j ) ) = 1 - \frac{edit ( s ( i ) , s ( j ) )}{max ( len ( s ( i ) ) , len ( s ( j ) ) )}$$

where len(⋅) represents the length (i.e., total number of characters) of a line-notation description such as a Structure Simplified Molecular Input Line Entry System (SMILES) representation.

In another example, a similarity metric value 110A may include a Longest Common Subsequence (LCS) value, which aims to find the longest common subsequence of characters between two strings. Embodiments of the invention may use it to detect sub-sequences of characters shared by two line-notation description (e.g., SMILES representations) s(i) and s(j). We denote LCS of s(i) and s(j)) by LCS (s(i); s(j)).

In another example, a similarity metric value 110A may include a Normalized Longest Common Subsequence (NLCS) value, which may be computed according to the following equation:

$$N L C S ( s ( i ) , s ( j ) ) = \frac{{len ( L C S ( s ( i ) , s ( j ) ) )}^{2}}{len ( s ( i ) ) \times len ( s ( j ) )}$$

In another example, a similarity metric value 110A may include a Term Frequency (TF) value. For example, embodiments of the invention may calculate a line-notation description (e.g., SMILES s(i)) with a vector composed of the frequency of each character Cₓ, referred to herein as TF(s(i)). Embodiments may subsequently calculate a cosine similarity metric value of TF(s(⋅)) according to the following equation:

$$Cosine ( s ( i ) , s ( j ) ) = \frac{T F ( s ( i ) ) \cdot T F ( s ( j ) )}{‖ T F ( s ( i ) ) ‖ ‖ T F ( s ( j ) ) ‖}$$

In another example, a similarity metric value 110A/110B may include an ensemble value. For example, embodiments of the invention may calculate an average of the predictions made with each of the abovementioned similarity metric values.

Embodiments of the invention may use SMILES to represent and recover the chemical structure for each drug. In SMILES, chemical atoms and bonds may be denoted by characters. In this method, one-hot encoding, and a gated recurrent unit (GRU) may be used with the SMILES representation.

Embodiments of the invention may represent each character in each SMILES representation with a one-hot encoding vector where each vector's size is equal to the number of unique characters in the dataset. Then, based on the one-hot encoding vectors, we utilize a GRU, which can process time-series information, capturing hidden patterns for different prediction tasks.

It may be appreciated that using a GRU based on consecutive characters' one-hot vectors may allow embodiments of the invention to capture hidden relations between different drugs' SMILES characters and leverage these connections to predict interactions between drugs.

Embodiments of the invention may use the same GRU for both drugs' SMILES and concatenate the hidden representation for the drugs' SMILES. Based on the concatenation of the SMILES hidden representation (i.e., the output of the GRU), embodiments may add a layer with a single unit and the sigmoid activation function to predict the DDIs.

Embodiments of the invention may use Char2Vec to represent characters in the SMILES representation with a latent vector. This method is motivated by the success of word2vec in representing words in a latent space. Using Char2Vec may enable embodiments of the invention to represent each character with respect to its context (e.g., the surrounding characters), while capturing different patterns in the chemical structure of various drugs.

Embodiments of the invention may thus utilize the GRU based on the SMILES characters' representations derived from Char2Vec.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

The following Table 1 includes a glossary of terms used herein, for the reader's convenience.

TABLE 1

| | |
|---|---|
| Neural Network | The term Neural Network (NN) or Artificial Neural Network |
| (NN), Artificial | (ANN), e.g., a neural network implementing a Machine Learning |
| Neural Network | (ML) or Artificial Intelligence (AI) function, may refer to an |
| (ANN) | information processing paradigm that may include nodes, referred |
| | to as neurons, organized into layers, with links between the |
| | neurons. The links may transfer signals between neurons and may |
| | be associated with weights. A NN may be configured or trained for |
| | a specific task, e.g., pattern recognition or classification. Training |
| | a NN for the specific task may involve adjusting these weights |
| | based on examples. Each neuron of an intermediate or last layer |
| | may receive an input signal, e.g., a weighted sum of output signals |
| | from other neurons, and may process the input signal using a linear |
| | or nonlinear function (e.g., an activation function). The results of |
| | the input and intermediate layers may be transferred to other |
| | neurons and the results of the output layer may be provided as the |
| | output of the NN. Typically, the neurons and links within a NN are |
| | represented by mathematical constructs, such as activation |
| | functions and matrices of data elements and weights. A processor, |
| | e.g., CPUs or graphics processing units (GPUs), or a dedicated |
| | hardware device may perform the relevant calculations. |
| Substance, | The terms “substance” and “drug” may be used herein |
| Substance of | interchangeably to indicate chemical compounds that may be |
| Interest, Drug | analyzed by embodiments of the invention to predict occurrence of |
| | DDI therebetween. A subtle difference in usage of these terms may |
| | serve to indicate that a “substance” or “substance of interest” may |
| | not yet be officially licensed as a drug (e.g., at a preclinical stage), |
| | and thus information regarding DDI with other drugs and |
| | compounds may be lacking. |
| Baseline, Baseline | The terms “baseline” and “baseline drug” may be used herein |
| drug | interchangeably to indicate a specific drug for which information |
| | regarding occurrence of DDIs, e.g., expected occurrence of |
| | chemical or biological interactions with other drugs or substances |
| | is already known. Such information may be applied by |
| | embodiments of the invention to predict occurrence of DDI with |
| | new SOIs and/or among SOIs, as elaborated herein. |
| Target drug, Target | The terms “target drug” and “target baseline drug” may be used |
| baseline drug | herein interchangeably to indicate a specific baseline drug of a |
| | plurality of baseline drugs. As elaborated herein, embodiments of |
| | the invention may be configured to predict occurrence of DDI |
| | between a specific target drug and one or more SOIs. |

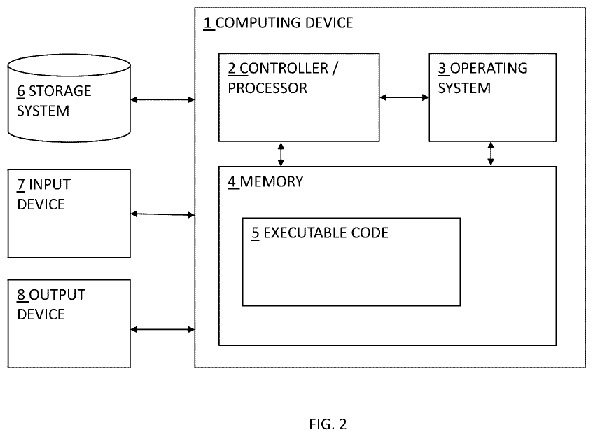

Reference is now made to FIG. 2, which is a block diagram depicting a computing device, which may be included within an embodiment of a system for predicting, or providing a prediction of DDI, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may predict DDI as further described herein. Although, for the sake of clarity, a single item of executable code 5 is shown in FIG. 2, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to inter-drug interaction may be stored in storage system 6 and may be loaded from storage system 6 into memory 4 where it may be processed by processor or controller 2. In some embodiments, some of the components shown in FIG. 2 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse, and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

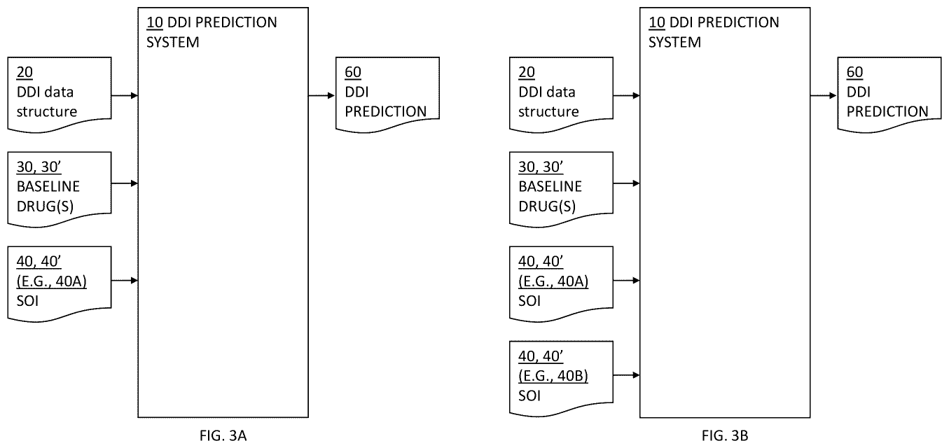

Reference is now made to FIGS. 3A and 3B, which are block diagrams depicting two exemplary applications of a system 10 for predicting DDI, according to some embodiments of the invention.

As shown in FIG. 3A, in some applications system 10 may receive, (e.g., from input device 7 of FIG. 2) a DDI data structure 20. DDI data structure 20 may, for example be formatted as a table, a linked list, a dictionary, a matrix, and the like, where each entry (i,j) may represent a known DDI between a first baseline drug (i) and a second baseline drug (j) of a plurality of baseline drugs.

According to some embodiments, DDI information of baseline drugs may be presented in DDI data structure 20. For example, a positive (e.g., ‘1’) value in entry (i,j) of DDI data structure 20 may indicate that a first baseline drug, associated with an index ‘i’ may be known, or expected to interact with a second baseline drug, associated with an index ‘j’. Additionally, a null (e.g., ‘0’) value in entry (i,j) of DDI data structure 20 may indicate that baseline drug ‘i’ may be known not to interact with baseline drug ‘j’. Additionally, a special value (e.g., Not Applicable (“N\A”)) in entry (i,j) of DDI data structure 20 may indicate that there is no specific information currently available to indicate whether baseline drug ‘i’ is expected to interact with baseline drug ‘j’. Additionally, or alternatively, a value in entry (i,j) of DDI data structure 20 may indicate a type, an extent, or an amplitude of a known or expected DDI between baseline drug ‘i’ and baseline drug ‘j’.

According to some embodiments, and as shown in FIG. 3A, system 10 may receive, e.g., from input device 7 of FIG. 2, a plurality of baseline drug data elements 30′. Baseline drug data elements 30′ may each correspond to, or be associated with a specific baseline drug 30 or baseline substance 30, that may be included in, or represented by an entry of DDI data structure 20. Each baseline drug data element 30′ may be, or may include a data structure (e.g., a string, a table, a linked list, etc.) that may describe a chemical structure of the corresponding baseline drug of the plurality of baseline drugs.

For example, a baseline drug data element 30′ may include a line-notation description, such as a Structure Simplified Molecular Input Line Entry System (SMILES) representation of the relevant, associated baseline drug. It may be appreciated by a person skilled in the art that a line-notation description of structure, such as the SMILES representation, may provide a simplified level of information that may be sufficient to convey or represent a chemical structure of a substance of interest at a basic, or primary level, but nevertheless be devoid of high-level vectorial representation of atoms and groups within the substance of interest.

As explained herein, the usage of simplified line-notation description of chemical structure for prediction of DDI occurrence may provide an improvement over currently available systems for drug analysis, which use large, elaborate data elements for chemical structure representation: It has been experimentally found that line-notation description of chemical structure may allow neural-network (NN) based models to generalize classification or prediction of DDI occurrence among drugs, without the risk of model overfitting due to extensive data input.

Additionally, or alternatively, system 10 may receive (e.g., from input 7 of FIG. 2) a substance data element 40′ (e.g., 40A′) that may correspond to, or be associated with a specific Substance of Interest (SOI) 40. Such SOI 40 may be, for example, a new drug or substance, for which occurrence of DDI with baseline drugs 30 (e.g., represented by baseline drug data elements 30′) of the plurality of baseline drugs 30 (e.g., included in DDI data structure 20) is to be predicted by system 10.

According to some embodiments, SOI data element 40′ (e.g., 40A′) may be, or may include a line-notation description of a chemical structure of a specific substance of interest 40. For example, SOI data element 40′ may include a SMILES representation of the new drug, as explained above.

Reference is now made to FIG. 4, which is a block diagram depicting modules of a system 10 for predicting DDI, according to some embodiments of the invention. System 10 of FIG. 4 may be the same as system 10 of FIG. 3A and/or FIG. 3B.

According to some embodiments of the invention, system 10 may be implemented as a software module, a hardware module, or any combination thereof. For example, system 10 may be, or may include a computing device such as element 1 of FIG. 2, and may be adapted to execute one or more modules of executable code (e.g., element 5 of FIG. 2) to produce a prediction of DDI 60, as further described herein.

As shown in FIG. 4, arrows may represent flow of one or more data elements to and/or from system 10 and/or among modules or elements of system 10. Some arrows have been omitted in FIG. 4 for the purpose of clarity.

As elaborated herein, system 10 may facilitate a similarity-based method of performing preclinical prediction of DDI occurrence involving new drugs of interest. The term “similarity-based” may be used in this context to indicate that methods of the present invention may not require intricate analysis of chemical structures of a new drug of interest, to ascertain occurrence of DDI between the SOI (e.g., a new drug) 40 and a specific target drug 50. Instead, system 10 may identify drugs that are structurally similar to the new drug of interest, for which occurrence (or lack thereof) of DDI with the target drug 50 is already known, and infer that knowledge on the new drug of interest 40.

As shown in FIG. 4, system 10 may include a similarity calculation module 110 (or similarity 110, for short), configured to calculate at least one similarity metric value 110A/110B, representing a structural similarity between a chemical structure of the substance of interest 40 (e.g., as represented by SOI 40′) and a chemical structure of a specific baseline drug 30 (e.g., as represented by the relevant baseline drug data element 30′).

For example, similarity module 110 may receive: (a) one or more baseline drug data elements 30′, that include SMILES representations of chemical structure of one or more respective baseline drugs, and (b) an SOI data element 40′ that includes a SMILES representation of a substance of interest 40 (e.g., a new drug, beyond the group of baseline drugs). Similarity module 110 may calculate one or more similarity metric values 110A, that represent similarity (or distance) between the substance of interest 40 (e.g., the new drug, represented by SOI data element 40) and the one or more baseline drugs 30 (e.g., represented by data element(s) 30′).

The one or more similarity metric values 110A may be, or may include for example a Tanimoto similarity value 110A, an edit-distance similarity value 110A, a Longest Common Subsequence (LCS) similarity value 110A, a Normalized Longest Common Subsequence (NLCS) similarity value 110A, a Term frequency (TF) similarity value 110A and the like.

For example, similarity metric module 110 may calculate an edit-distance similarity value 110A by calculating an edit value 110A′, representing a number of edit operations that are required in order to convert the SOI (e.g., new drug) data element 40′ to a relevant baseline drug data element 30′. Similarity metric module 110 may subsequently normalize the edit value 110A′ by a size or length (e.g., a number of symbols) of the relevant SOI 40′ (e.g., new drug) data element, to produce the edit-distance similarity value 110A. Additionally, or alternatively, similarity metric module 110 may normalize the edit value 110A′ by a size or length (e.g., a number of symbols) of relevant baseline drug data element 30′, to produce the edit-distance similarity value 110A.

Additionally, or alternatively, similarity metric module 110 may calculate one or more composite similarity metric values 110B. For example, similarity metric module 110 may calculate two or more first, or interim similarity metric values 110A (e.g., a Tanimoto similarity value, an edit distance similarity value, an LCS similarity value, an NLCS similarity value, a TF similarity value, etc.) based on the SMILES representations of the specific baseline drug data element and the first substance data element. Similarity metric module 110 may subsequently calculate a second, or composite similarity metric value 110B as a function (e.g., a weighted average) of the two or more first similarity metric values 110A.

According to some embodiments, DDI prediction system 10 may include a selection module 120, configured to select a subset 120A or a number 120A of the plurality of baseline drugs represented in DDI data structure 20, based on the one or more first similarity metric values. For example, selection module 120 may compare the similarity metric values 110A/110B associated with a plurality of baseline drug data elements 30′, and select a number (e.g., 3) baseline drug data elements 30′ that are most similar (e.g., have the highest similarity metric values 110A/110B) in relation to the SOI 40 of SOI data element 40′. selection module 120 may represent the selected subset 120A by identification (e.g., a name, a serial number, etc.) of the relevant baseline drugs 30.

According to some embodiments, system 10 may include a machine-learning (ML) based module 130. ML module 130 may be configured to receive an identification of at least one target baseline drug 50 of the plurality of baseline drugs 30, and predict occurrence of DDI between target drug 50 and the substance of interest 40. In other words, ML module 130 may be adapted to apply a machine-learning algorithm to predict occurrence of DDI between SOI 40 and a target baseline drug 50 of the plurality of baseline drugs, based on the selected subset 120A of baseline drugs and (b) DDI data structure 20, as elaborated herein.

According to some embodiments, ML module 130 may receive as input: (i) identification of the selected subset 120A of baseline drug data elements 30′, and (ii) at least a portion of DDI data structure 20. The portion of DDI data structure 20 may include representation of the selected baseline drugs 30 (e.g., corresponding to the selected subset 120A of baseline drug data elements 30′). Based on this received input, ML module 130 may be configured to produce a prediction 60 of occurrence of DDI between the substance of interest 40 (represented by SOI 40′) and at least one specific baseline drug 30 (represented by data element 30′) or target drug 50 of the plurality of baseline drugs 30, as elaborated herein.

According to some embodiments, prediction 60 may be a Boolean numeric value, where ‘1’ may represent that DDI is expected to occur between the substance of interest 40 and the at least one specific baseline drug 30, and ‘0’ may represent that no such DDI is expected to occur between these substances. Additionally, or alternatively, prediction 60 may be a Real numeric value (e.g., in the range of [0, 1]), representing a probability that such DDI would occur between these substances.

As known in the art, an “embedding” or “embedding space” is a relatively low-dimensional space into which information conveyed by a high-dimensional vector may be translated. Embeddings are typically applied by machine learning algorithms on large, optionally sparse inputs vectors, to represent these vectors in a concise manner, while maintaining information that is pertinent to the underlying machine-learning task.

According to some embodiments, ML module 130 may include an embedding module 133, configured to produce at least one DDI embedding vector 134 (e.g., 134A, 134B, 13C). DDI embedding vector 134 may represent, in an embedding space, information of DDI data structure 20 that pertains to at least one specific baseline drug 30.

For example, embedding module 133 may be configured to receive (e.g., from processor 1 of FIG. 2) an identification, or reference to a specific baseline drug 30, and extract a vector (e.g., a column or row) of DDI data structure 20 that includes DDI information pertaining to that specific baseline drug 30. For example, DDI data structure 20 may be formatted as a matrix, where a column (*,i) or row (i,*) of DDI matrix 20 may be an interim vector that may include all known or expected DDIs between a specific baseline drug 30 (who's index is ‘i’) and all other baseline drugs 30 of DDI matrix 20. Embedding module 133 may then apply an embedding algorithm as known in the art on the extracted column or row (e.g., the interim vector) to obtain DDI embedding vector 134 pertaining to the specific, identified baseline drug 30.

As elaborated herein, ML module 130 may receive an identification (e.g., a name, a serial number, etc.) of a subset 120A of baseline drugs 30, which are determined by similarity module 110 as most structurally similar to that of SOI 40. ML module 130 may also receive an identification (e.g., a name, a serial number, etc.) of a target drug 50, for which DDI occurrence with SOI 40 is to be predicted.

As elaborated herein, embedding module 133 may extract, or calculate from DDI data structure 20 a first DDI embedding vector 134A, representing known DDIs between a baseline drug 30 of selected subset 120A and other baseline drugs 30 of the plurality of baseline drugs 30 in an embedding space. Additionally, or alternatively, embedding module 133 may extract, or calculate from DDI data structure 20 a second DDI embedding vector 134B, representing known DDIs between a target baseline drug 50 and other baseline drugs of the plurality of baseline drugs in the embedding space.

According to some embodiments, ML module 130 may include a vector manipulation module 135, configured to apply a vector operation on DDI embedding vector 134A and DDI embedding vector 134B. For example, vector manipulation module 135 may apply an element-wise multiplication of DDI embedding vector 134A and DDI embedding vector 134B to obtain a product DDI embedding vector 135A. Product DDI embedding vector 135A may represent a combination of DDI information that pertains to both the target drug 50 and the relevant baseline drug 30 of subset 120A. Additional implementations of vector manipulation module 135 and product DDI embedding vector 135A may also be possible.

It may be appreciated that DDI data structure 20 may be represented as a sparse matrix, meaning that DDI information may be lacking for some pairs or combinations of baseline drugs. As shown in FIG. 4, ML module 130 may include a NN-based ML model, denoted as NN 137, adapted to overcome this lack of information, as elaborated herein.

According to some embodiments, NN 137 may be configured to receive as input a product DDI embedding vector 135A from vector manipulation module 135. Additionally, or alternatively, NN 137 may be configured to receive as input the DDI embedding vector 134A and DDI embedding vector 134B from embedding module 133. NN 137 may be trained to calculate, based on this input, a DDI score 137A, representing expectancy or probability of DDI occurrence between the relevant baseline drugs 30.

For example, DDI score 137A may be a numerical value (e.g., in the range of [0, 1]), representing an expected DDI between a baseline drug 30 of selected subset 120A (e.g., represented by DDI embedding vector 134A) and a target baseline drug 50 (e.g., represented by DDI embedding vector 134B).

As shown in FIG. 4, ML module 130 may include a decision module 139, configured to produce a prediction 60, representing expected occurrence of DDI between SOI 40 and a target baseline drug 50 based on the DDI scores 137.

As elaborated herein, ML module 130 may infer NN model 137 on product DDI embedding vector 135A. ML module 130 may thereby apply a machine-learning algorithm on DDI embedding vector 134A and DDI embedding vector 134B, to calculate a DDI scores 137A, based on DDI data structure 20.

DDI score 137A may represent expectancy or probability of DDI occurrence between a baseline drug 30 of subset 120A and target baseline drug 50. ML module 130 may repeat this process for one or more (e.g., all) baseline drugs 30 of subset 120A, to obtain one or more respective DDI scores 137A.

Decision module 139 may subsequently analyze the one or more DDI scores 137A to produce prediction 60, representing an expected DDI between SOI 40 and target baseline drug 50. For example, decision module 139 may compare a first number of DDI scores 137A that surpass a predefined threshold to a second number of DDI scores 137A that fall below the predefined threshold, to provide a Boolean prediction 60 based on this comparison (e.g., when the first counted number surpasses the second counted number). Additional decision algorithms may also be available.

As shown in FIG. 3B, system 10 may receive (e.g., from input 7 of FIG. 2) two substance data elements 40′, e.g., 40A′ and 40B′, each corresponding to, or associated with a specific SOI 40, e.g., 40A and 40B. SOIs 40A and 40B may be, for example, two new drugs or substances, for which occurrence of DDI therebetween is to be predicted by system 10.

The process of predicting 60 occurrence of DDI between two SOIs 40 may be similar to the process of predicting 60 occurrence of DDI between an SOI 40 and a baseline drug 30 (or target baseline drug 50), as elaborated herein, and will not be repeated herein in its entirety, for the purpose of brevity.

As elaborated herein in relation to a single SOI data element 40A′, the second substance data element 40B′ may include a line-notation description of a chemical structure of the second SOI 40B. Similarity module 110 may thus calculate one or more similarity metric values 110A/110B, each representing a structural similarity between the second substance of interest 40B and a specific baseline drug 30, based on substance data element 40B′ and the relevant baseline drug data element 30′. Selection module 120 may then select a second subset 120B of the plurality of baseline drugs 30, based on the one or more second similarity metric values. For example, selection module 120 may select baseline drug data elements 30′ that are most similar (e.g., have the highest similarity metric values 110A/110B) in relation to the second SOI 40 of SOI data element 40′.

ML module 130 may subsequently predict 60 occurrence of DDI between the first SOI 40A and the second SOI 40B, based on (a) the first subset 120A of baseline drugs, (b) the second subset 120B of baseline drugs, and (c) DDI data structure 20, as elaborated herein.

As elaborated herein in relation to a single SOI data element 40A′, embedding module 133 may extract, or calculate from DDI data structure 20 a DDI embedding vector 134A, representing known DDIs between a baseline drug 30 of subset 120A and other baseline drugs 30 of the plurality of baseline drugs 30. Additionally, or alternatively, embedding module 133 may extract, or calculate from DDI data structure 20 a DDI embedding vector 134C, representing known DDIs between a baseline drug 30 of the second subset 120B and other baseline drugs 30 of the plurality of baseline drugs 30 in DDI data structure 20. Vector manipulation module 135 may subsequently apply a vector operation on DDI embedding vector 134A and DDI embedding vector 134C, to obtain a product DDI embedding vector 135B. Product DDI embedding vector 135B may represent a combination of DDI information that pertains to both the baseline drug 30 of subset 120A and the baseline drug 30 of subset 120B.

As elaborated herein in relation to a single SOI data element 40A′, ML module 130 may infer NN model 137 on product DDI embedding vector 135B. ML module 130 may thereby apply a machine-learning algorithm on DDI embedding vector 134A and DDI embedding vector 134B, to calculate a DDI score 137B, based on DDI data structure 20.

DDI score 137B may represent expectancy or probability of DDI occurrence between a baseline drug 30 of subset 120A and a baseline drug 30 of subset 120B. ML module 130 may repeat this process for one or more (e.g., all) baseline drugs 30 of subset 120A and subset 120B, to obtain one or more respective DDI scores 137B.

Decision module 139 may subsequently analyze the one or more DDI scores 137B to produce prediction 60. In such embodiments, prediction 60 may representing an expected DDI between the first SOI 40A and the second SOI 40B. For example, decision module 139 may compare a first number of DDI scores 137B that surpass a predefined threshold to a second number of DDI scores 137B that fall below the predefined threshold, to provide a Boolean prediction 60 based on this comparison (e.g., when the first counted number surpasses the second counted number). Additional decision algorithms may also be available.

According to some embodiments, system 10 may include a regimen module 138, adapted to produce a regimen recommendation data element 70, based on the predicted occurrence of DDI 60. For example, regimen module 138 may analyze DDI predictions 60 of SOI 40 in view of a group of baseline drugs 30 (e.g., baseline drugs 30 that have similar modes-of-action, target and/or indication). Regimen module 138 may subsequently determine a recommended drug regimen 70 that includes at least the SOI 40, based on the predicted occurrence of DDI 60. For example, regimen module 138 may select (a) the relevant SOI 40, and (b) zero, one or more baseline drugs 30 of the group of baseline drugs 30 that are predicted 60 not to interact with the SOI 40 or predicted 60 to only weakly interact (e.g., below a predefined threshold) with SOI 40, to be included in regimen 70. Additionally, or alternatively, regimen module 138 may avoid including baseline drugs 30 that are predicted 60 to interact with the SOI 40 into regimen 70.

System 10 may subsequently produce a regimen recommendation 70′ data element, that may represent or include the determined regimen 70, and may transmit regimen recommendation 70′ data element, for example as an electronic message (e.g., an email message) to at least one computing device (e.g., computing device 1 of FIG. 2) of a predefined user.

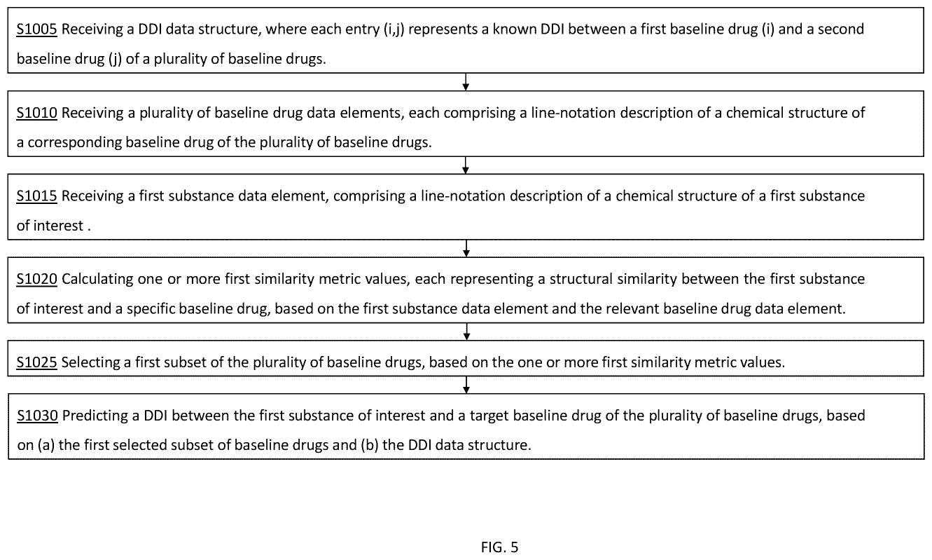

Reference is now made to FIG. 5 which is a flow diagram, depicting a method of predicting occurrence of DDI by at least one processor (e.g., processor 2 of FIG. 2), according to some embodiments of the invention.

As shown in step S1005, the at least one processor 2 may receive a DDI data structure (e.g., DDI data structure 20 of FIGS. 3A, 3B and 4), wherein each entry (i,j) may represent a known or expected DDI between a first baseline drug 30 (i) (e.g., element 30 of FIG. 4) and a second baseline drug 30 (j) of a plurality of baseline drugs 30.

As shown in step S1010, the at least one processor 2 may receive a plurality of baseline drug data elements 30′, each including a line-notation description of a chemical structure of a corresponding baseline drug 30 of the plurality of baseline drugs 30.

Additionally, or alternatively, and as shown in step S1015, the at least one processor 2 may receive a substance data element 40′ that includes a simplified, line-notation description of a chemical structure of respective substance of interest.

As shown in step S1020, the at least one processor 2 may employ a similarity module (e.g., similarity module 110 of FIG. 4) to calculate one or more first similarity metric values (e.g., elements 110A/110B of FIG. 4), each representing a structural similarity between the substance of interest 40 and a specific baseline drug 30, based on the first substance data element 40′ and the relevant baseline drug data element 30′.

As shown in step S1025, the at least one processor 2 may employ a selection module (e.g., selection module 120 of FIG. 4) to select a subset (e.g., element 120A of FIG. 4) of the plurality of baseline drugs 30, based on the calculated one or more first similarity metric values 110A/110B.

As shown in step S1025, the at least one processor 2 may employ an ML module (e.g., element 130 of FIG. 4) to predict occurrence of DDI between the substance of interest 40 and a target baseline drug 50 of the plurality of baseline drugs 30, based on (a) the selected subset 120A of baseline drugs 30 and (b) the DDI data structure 20.

As elaborated herein, embodiments of the invention may provide a practical application for predicting occurrence of DDIs between drugs, and may thus facilitate a plurality of improvements over currently available methods and systems for drug manufacturing in the technological field of pharmaceutics.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

What is claimed is:

1. A method of predicting drug-drug interactions (DDIs) by at least one processor, the method comprising:
receiving a DDI data structure, wherein each entry (i,j) represents a known DDI between a first baseline drug (i) and a second baseline drug (j) of a plurality of baseline drugs;
receiving a plurality of baseline drug data elements, each comprising a line-notation description of a chemical structure of a corresponding baseline drug of the plurality of baseline drugs;
receiving a first substance data element, comprising a line-notation description of a chemical structure of a respective first substance of interest;
calculating one or more first similarity metric values, each representing a structural similarity between the first substance of interest and a specific baseline drug, based on the first substance data element and the relevant baseline drug data element;
selecting a first subset of the plurality of baseline drugs, based on the one or more first similarity metric values; and
predicting occurrence of DDI between the first substance of interest and a target baseline drug of the plurality of baseline drugs, based on (a) the first selected subset of baseline drugs and (b) the DDI data structure.

2. The method of claim 1, further comprising
receiving a second substance data element, comprising a line-notation description of a chemical structure of a second substance of interest;
calculating one or more second similarity metric values, each representing a structural similarity between the second substance of interest and a specific baseline drug, based on the second substance data element and the relevant baseline drug data element;
selecting a second subset of the plurality of baseline drugs, based on the one or more second similarity metric values; and
predicting a DDI between the first substance of interest and the second substance of interest, based on (a) the first subset of baseline drugs, (b) the second subset of baseline drugs, and (c) the DDI data structure.

3. The method of claim 1, wherein the similarity metric values are selected from a Tanimoto similarity value, an edit distance similarity value, a Longest Common Subsequence (LCS) similarity value, a Normalized Longest Common Subsequence (NLCS) similarity value, and a Term frequency (TF) similarity value.

4. The method of claim 1, wherein calculating a similarity metric value between a new drug and a baseline drug comprises:
calculating an edit value, representing a number of edit operations that are required in order to convert the new drug data element to the baseline drug data element; and
normalizing the edit value by at least one of a length of the new drug data element and the baseline drug data element.

5. The method of claim 1, wherein the line-notation description comprises a Structure Simplified Molecular Input Line Entry System (SMILES) representation.

6. The method of claim 5, wherein calculating a similarity metric value between the first substance of interest and a specific baseline drug comprises:
calculating two or more first similarity metric values, selected from a group consisting of: a Tanimoto similarity value, an edit distance similarity value, an LCS similarity value, an NLCS similarity value, and a TF similarity value, based on the SMILES representations of the specific baseline drug data element and the first substance data element; and
calculating a second similarity metric value, as a function of the two or more first similarity metric values.

7. The method of claim 1, wherein predicting a DDI between the first substance of interest and the target baseline drug comprises:
applying a machine-learning algorithm to calculate one or more DDI scores, representing an expected DDI between one or more baseline drugs of the first subset of baseline drugs, and the target baseline drug, based on the DDI data structure; and
predicting DDI between the first substance of interest and the target baseline drug based on the one or more DDI scores.

8. The method of claim 1, wherein predicting a DDI between the first substance of interest and the second substance of interest comprises:
applying a machine-learning algorithm to calculate one or more DDI scores, representing an expected DDI between one or more baseline drugs of the first subset of baseline drugs, and one or more respective baseline drugs of the second subset of baseline drugs, based on the DDI data structure; and
predicting DDI between the first substance of interest and the second substance of interest based on the one or more DDI scores.

9. The method of claim 1, wherein calculating a DDI score representing an expected DDI between a first baseline drug and a second baseline drug comprises:
extracting, from the DDI data structure, a first DDI embedding vector, representing known DDIs between the first baseline drug and other baseline drugs of the plurality of baseline drugs in an embedding space;
extracting, from the DDI data structure, a second DDI embedding vector, representing known DDIs between the second baseline drug and other baseline drugs of the plurality of baseline drugs in the embedding space;
applying a vector operation on the first DDI embedding vector and the second DDI embedding vector, to produce a DDI embedding vector; and
applying a machine-learning model on the product DDI embedding vector, to obtain the DDI score.

10. The method of claim 9, wherein extracting a DDI embedding vector of a specific baseline drug from the DDI data structure comprises:
extracting an interim vector of the DDI data structure that corresponds to the specific baseline drug; and
applying an embedding algorithm on the extracted interim vector to obtain the DDI embedding vector of the specific baseline drug.

11. The method of claim 1 further comprising:
determining a drug regimen, comprising at least the first substance of interest, based on the predicted occurrence of DDI; and
producing a regimen recommendation data element, representing the determined regimen.

12. A system for predicting DDIs, the system comprising: a non-transitory memory device, wherein modules of instruction code are stored, and at least one processor associated with the memory device, and configured to execute the modules of instruction code, whereupon execution of said modules of instruction code, the at least one processor is configured to:
receive a DDI data structure, wherein each entry (i,j) represents a known DDI between a first baseline drug (i) and a second baseline drug (j) of a plurality of baseline drugs;
receive a plurality of baseline drug data elements, each comprising a line-notation description of a chemical structure of a corresponding baseline drug of the plurality of baseline drugs;
receive a first substance data element, comprising a line-notation description of a chemical structure of a first substance of interest;
calculate one or more first similarity metric values, each representing a structural similarity between the first substance of interest and a specific baseline drug, based on the first substance data element and the relevant baseline drug data element;
select a first subset of the plurality of baseline drugs, based on the one or more first similarity metric values; and
predict occurrence of DDI between the first substance of interest and a target baseline drug of the plurality of baseline drugs, based on (a) the first selected subset of baseline drugs and (b) the DDI data structure.

13. The system of claim 12, wherein the at least one processor is further configured to:
receive a second substance data element, comprising a line-notation description of a chemical structure of a second substance of interest;
calculate one or more second similarity metric values, each representing a structural similarity between the second substance of interest and a specific baseline drug, based on the second substance data element and the relevant baseline drug data element;
select a second subset of the plurality of baseline drugs, based on the one or more second similarity metric values; and
predict a DDI between the first substance of interest and the second substance of interest, based on (a) the first subset of baseline drugs, (b) the second subset of baseline drugs, and (c) the DDI data structure.

14. The system of claim 12, wherein the similarity metric values are selected from a Tanimoto similarity value, an edit distance similarity value, a Longest Common Subsequence (LCS) similarity value, a Normalized Longest Common Subsequence (NLCS) similarity value, and a Term frequency (TF) similarity value.

15. The system of claim 12, wherein the at least one processor is further configured to calculate a similarity metric value between a new drug and a baseline drug by:
calculating an edit value, representing a number of edit operations that are required in order to convert the new drug data element to the baseline drug data element; and
normalizing the edit value by at least one of a length of the new drug data element and the baseline drug data element.

16. The system of claim 12, wherein the line-notation description comprises a Structure Simplified Molecular Input Line Entry System (SMILES) representation.

17. The system of claim 16, wherein the at least one processor is further configured to calculate a similarity metric value between the first substance of interest and a specific baseline drug by:
calculating two or more first similarity metric values, selected from a group consisting of: a Tanimoto similarity value, an edit distance similarity value, an LCS similarity value, an NLCS similarity value, and a TF similarity value, based on the SMILES representations of the specific baseline drug data element and the first substance data element; and
calculating a second similarity metric value, as a function of the two or more first similarity metric values.

18. The system of claim 12, wherein the at least one processor is further configured to predict a DDI between the first substance of interest and the target baseline drug by:
applying a machine-learning algorithm to calculate one or more DDI scores, representing an expected DDI between one or more baseline drugs of the first subset of baseline drugs, and the target baseline drug, based on the DDI data structure; and
predicting DDI between the first substance of interest and the target baseline drug based on the one or more DDI scores.

19. The system of claim 12, wherein the at least one processor is further configured to predict a DDI between the first substance of interest and the second substance of interest by:
applying a machine-learning algorithm to calculate one or more DDI scores, representing an expected DDI between one or more baseline drugs of the first subset of baseline drugs, and one or more respective baseline drugs of the second subset of baseline drugs, based on the DDI data structure; and
predicting DDI between the first substance of interest and the second substance of interest based on the one or more DDI scores.

20. The system of claim 12, wherein the at least one processor is further configured to calculate a DDI score representing an expected DDI between a first baseline drug and a second baseline drug by:
extracting, from the DDI data structure, a first DDI embedding vector, representing known DDIs between the first baseline drug and other baseline drugs of the plurality of baseline drugs in an embedding space;
extracting, from the DDI data structure, a second DDI embedding vector, representing known DDIs between the second baseline drug and other baseline drugs of the plurality of baseline drugs in the embedding space;
applying a vector operation on the first DDI embedding vector and the second DDI embedding vector, to produce a DDI embedding vector; and
applying a machine-learning model on the product DDI embedding vector, to obtain the DDI score.

21.-22. (canceled)
